(19) United States
(12) Patent Application Publication
(10) Pub. No.: US 2024/0035034 A1
(43) Pub. Date: Feb. 1, 2024

(54) USE OF A COMBINATION OF AN ORPHAN MOTIF AND CPG DENSITY TO CONTROL EXPRESSION OF A HETEROLOGOUS TRANSGENE

(71) Applicant: Friedrich Miescher Institute for Biomedical Research, Basel (CH)

(72) Inventors: Ralph GRAND, Basel (CH); Marlena LUEBKE, Basel (CH); Dirk Schuebeler, Basel (CH)

(21) Appl. No.: 18/255,296

(22) PCT Filed: Dec. 2, 2021
(86) PCT No.: PCT/IB2021/061228

§ 371 Date: May 31, 2023

(30) Foreign Application Priority Data
Dec. 3, 2020 (EP) 20211437.7

(51) Int. Cl. C12N 15/113; C07K 14/725; C07K 14/47

(52) U.S. Cl. CPC C12N 15/1138; C07K 14/7051; C07K 14/4703

(57) ABSTRACT

The present invention provides an isolated nucleic acid comprising more than 220 bp, one or more copy of a sequence selected from the group of SEQ ID NO:1, SEQ ID NO:2 and SEQ ID NO:3, and a CpG Observed over Estimated ratio (O/E ratio) larger than 0.6 in the N base pairs (bp) preceding and/or in the N bp following said one or more copy of a sequence selected from the group of SEQ ID NO:1, SEQ ID NO:2 and SEQ ID NO:3, wherein the CpG O/E ratio is determined by counting the number of CpG dinucleotides in the N bp-long sequences surrounding the at least one or more copy of a sequence selected from the group of SEQ ID NO:1, SEQ ID NO:2 and SEQ ID NO:3 and calculating the O/E ratio by multiplying the counted number of CpG dinucleotides by N and dividing the result by the product of the number of C and number of G present in the N bp (N*CpG/(C*G)), wherein N is between 50 and 1000 and is the length, in bp, of the sequence immediately preceding or immediately following said one or more copy of a sequence selected from the group of SEQ ID NO:1, SEQ ID NO:2 and SEQ ID NO:3.

### FIELD OF THE INVENTION

The present invention relates to a nucleic acid sequence leading to the controlled expression of heterologous transgenes operatively linked to it.

### BACKGROUND OF THE INVENTION

Gene therapy methods that deliver genetic material (e.g., heterologous nucleic acids) into target cells in order to increase the expression of desired gene products support therapeutic objectives. Viruses have evolved to become highly efficient at nucleic acid delivery to specific cell types while avoiding immunosurveillance by an infected host (Robbins et al., (1998) Pharmacol. Ther., 80(1):35-47). These properties make viruses attractive as delivery vehicles, or vectors, for gene therapy. Several types of viruses, including retrovirus, adenovirus, adeno-associated virus (AAV), and herpes simplex virus, have been modified in the laboratory for use in gene therapy applications (Lunstrom et al., (2018) Diseases, 6(2): 42). In particular, vectors derived from Adeno-Associated Viruses (AAVs) may effectively deliver genetic material because (i) they are able to infect (transduce) a wide variety of non-dividing and dividing cell types including muscle fibers and neurons; (ii) they are devoid of the virus structural genes, thereby eliminating the natural host cell responses to virus infection, e.g., interferon-mediated responses; (iii) wild-type viruses have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response, thus granting long-term expression of, e.g., therapeutic heterologous nucleic acid(s) (Wold et al., (2013) Curr. Gene Ther., 13(6):421-33; Lee et al., (2017) Genes Dis., 4(2): 43-63). AAV is a member of the parvoviridae family. The AAV genome comprises a linear single-stranded DNA molecule which typically contains approximately 4.7 kilobases (kb) and two major open reading frames encoding the non-structural Rep (replication) and structural Cap (capsid) proteins. Flanking the AAV coding regions are two cis-acting inverted terminal repeat (ITR) sequences, which are typically approximately 145 nucleotides in length and have interrupted palindromic sequences that can fold into hairpin structures that function as primers during initiation of DNA replication. In addition to their role in DNA replication, the ITR sequences have been shown to contribute to viral integration, rescue from the host genome, and encapsidation of viral nucleic acid into mature virions (Muzyczka et al., (1992) Curr. Top. Micro. Immunol., 158:97-129).

While AAVs are desirable for their ability to transduce a variety of cell types and deliver the heterologous nucleic acids to a variety of target tissue types, delivery of the heterologous nucleic acids to tissue where expression of the heterologous nucleic acids is not needed, as well as high expression of the transgene where needed, remain a challenge. Careful calibration of gene expression in desired tissues may provide therapeutic benefits. AAV vectors containing CAG promoter have been used in a number of clinical trials of gene therapy, e.g. for CNS diseases (Hoequemiller et al., (2016) Hum. Gene Ther., 27(7): 478-96). There remains a need to develop methods of obtaining high expression of heterologous nucleic acids in specific tissues. There is thus a need for improved tissue-specific expression of therapeutic proteins (such as antibodies or functional binding fragments, enzymes, etc.), and of nucleic acids (such as shRNA, siRNA, gRNA for use in CRISPR, etc.).

Another barrier to more widespread use of viral vectors for gene delivery is the packaging capability of the vectors. For example, AAV vector genomes are typically limited to about 4.7 kb for the single stranded (ssAAV) and 2.4 kb for the self-complimentary (scAAV) vectors, which puts a limit on the size of the genetic payload that can be delivered (Wu et al., (2010) Mol. Ther., 18(1):80-86). Since the genetic payload includes regulatory elements, e.g., promoters, termination signals, etc., this further restricts the size of the heterologous nucleic acid that may be packaged. Thus, there is a need to provide regulatory elements of reduced length in order to allow insertion of heterologous nucleic acid sequences encoding larger proteins, e.g., in AAV derived vectors used in gene therapy.

### SUMMARY OF THE INVENTION

The present inventors have previously found that a thus far orphaned regulatory motif in mammalian, when bound by protein BANP, acts as a strong transcriptional activator, also of CpG island promoters. This strong activator effect is synergistically increased when more than one copy of the motif is present in front of a heterologous transgene.

Upon further investigations, the present inventors found that the number of CpG sites in the vicinity of the orphaned regulatory motif influences the activity of the motif. This effect can be used to regulate the expression of genes operatively linked to said motif. For example, an expression vector can harbor more than one heterologous transgene, each under the control of its respective BANP motif, but with a different CpG density around each of these motives. This will result in a different, controlled, expression of each of the transgenes, despite being on the same vector and controlled by the same motif bound by the same transcription factor. Another advantage of the present invention is that CpG-rich motives are usually highly controlled by the cells and will lead to a switching off of the expression of the transgene if the construct is accidentally incorporated into the genome of the host cell.

The present invention hence provides an isolated nucleic acid molecule comprising more than 220 bp, one or more copy of a sequence selected from the group of SEQ ID NO:1, SEQ ID NO:2 and SEQ ID NO:3, and a CpG Observed over Estimated ratio (O/E ratio) larger than 0.6 in the N base pairs (bp) preceding and/or in the N bp following said one or more copy of a sequence selected from the group of SEQ ID NO:1, SEQ ID NO:2 and SEQ ID NO:3, wherein the CpG O/E ratio is determined by counting the number of CpG dinucleotides in the N bp-long sequences surrounding the at least one or more copy of a sequence selected from the group of SEQ ID NO:1, SEQ ID NO:2 and SEQ ID NO:3 and calculating the O/E ratio by multiplying the counted number of CpG dinucleotides by N and dividing the result by the product of the number of C and number of G present in the N bp (N*CpG/(C*G)), wherein N is between 50 and 1000 and is the length, in bp, of the sequence immediately preceding or immediately following said one or more copy of a sequence selected from the group of SEQ ID NO:1, SEQ ID NO:2 and SEQ ID NO:3. For the sake of clarity, it is stated that it is well known to the skilled person that C stands for a cytosine nucleotide, G stands for guanosine nucleotide and CpG (or CG) stands for 5′-C-phosphate-G-3′, i.e. cytosine and guanine separated by only one phosphate group (phosphate links any two nucleosides together in DNA).

In some embodiments, the CpGs are present in the 50-1000 bp immediately preceding the one or more copy of a sequence selected from the group of SEQ ID NO:1, SEQ ID NO:2 and SEQ ID NO:3, and an heterologous transgene is situated, immediately or not, after said one or more copy of the sequence selected from the group of SEQ ID NO:1, SEQ ID NO:2 and SEQ ID NO:3.

In some embodiments, the CpGs are present in the 50-1000 bp immediately following the one or more copy of a sequence selected from the group of SEQ ID NO:1, SEQ ID NO:2 and SEQ ID NO:3.

In some embodiments, In some embodiments, the CpGs are present in the 50-1000 bp immediately preceding and on the 50-1000 bp following the one or more copy of a sequence selected from the group of SEQ ID NO:1, SEQ ID NO:2 and SEQ ID NO:3.

In some embodiments, N is approximatively 50. In some embodiments, N is approximatively 100. In some embodiments, N is approximatively 150. In some embodiments, N is approximatively 200. In some embodiments, N is approximatively 250. In some embodiments, N is approximatively 500. In some embodiments, N is approximatively 800. In some embodiments, N is approximatively 1000.

The nucleic acid sequence of the sequence of the invention are:
SEQ ID NO:1 BMYCGCGRBV
SEQ ID NO:2 YMYCGCGRKV
SEQ ID NO:3 TCTCGCGAGA

In some embodiments, the isolated nucleic acid of the invention further comprises, operably linked to a constitutive promoter or to an inducible promoter, a further sequence encoding for protein BANP, or for an active fragment or variant thereof.

In some embodiments, the heterologous transgene of the isolated nucleic acid of the invention is a chimeric antigen receptor.

The present invention also provides a vector comprising the isolated nucleic acid of the invention. In some embodiments, this vector is a plasmid, DNA vector, RNA vector, viral vector, adenoviral vector, adenoassociated viral vector, lentiviral vector, retroviral vector, gamma retroviral vector, or HSV vector. In some embodiments, the isolated nucleic acid of the invention is less than 8 Kb. In some embodiments, the isolated nucleic acid of the invention is less than 5 Kb.

The present invention also provides a kit or composition comprising an isolated nucleic acid of the invention and a second isolated nucleic molecule comprising a sequence encoding for protein BANP, or for an active fragment or variant thereof, operably linked to a constitutive promoter or to an inducible promoter. In such a kit, the isolated nucleic acids of the invention can be either within the same vector or within different vectors.

The present invention also provides the use of an isolated nucleic acid of the invention, a vector of the invention or a kit of the invention or composition of the invention for the, preferably transient, expression in vitro, ex vivo or in vivo of the heterologous transgene in a cell. In some embodiments, this use increases the expression of the heterologous transgene by a factor greater than two as compared to the expression of the heterologous transgene when operatively linked to a single copy of SEQ ID NO:1, SEQ ID NO:2 or SEQ ID NO:3 under the same conditions. In some embodiments, the expression of the heterologous transgene is measured by reporter gene activity, reporter gene fluorescence, quantitative reverse transcriptase PCR or genomics approaches such as RNA sequencing.

The present invention further provides a method of producing, in vitro, ex vivo or in vivo, a heterologous transgene in a cell by introducing any of the isolated nucleic acids of the invention or the vectors of the invention of claims the cell, culturing this cell (or cell population), and purifying the recombinantly expressed heterologous transgene. In some embodiments, the cell is a stem cell.

The present invention also provides the isolated cell comprising the isolated nucleic acid of the invention. In this cell, or cells, the isolated nucleic acid sequence comprising at least two copies of a sequence selected from the group of SEQ ID NO:1, SEQ ID NO:2 and/or SEQ ID NO:3 and the heterologous transgene can be stably integrated into the genome of said cell.

### BRIEF DESCRIPTION OF THE DRAWINGS

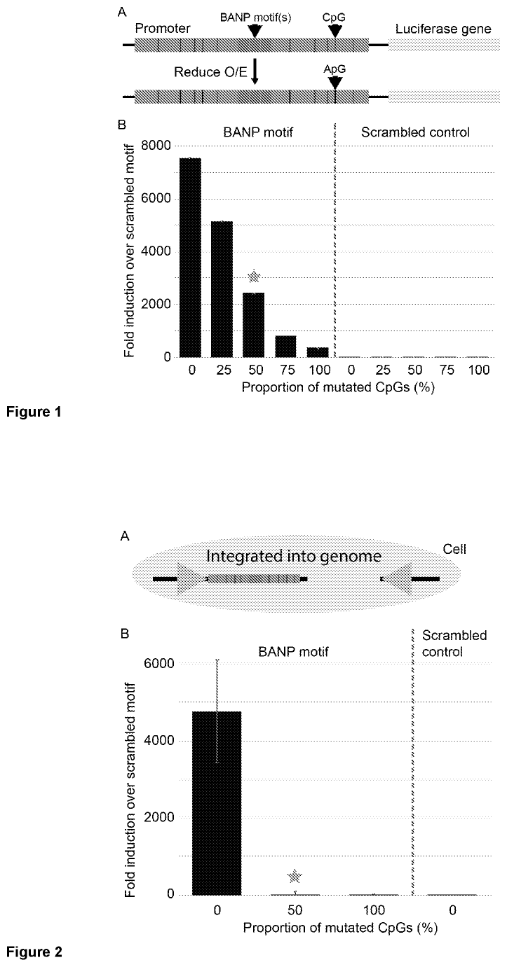

FIG. 1: Luciferase reporter activity can be tuned by adjusting the CpG density of an artificial Banp promoter
A) Banp motif(s) cloned upstream of Firefly Luciferase reporter gene indicating CpG dinucleotides in the surrounding artificial promoter sequence and their mutation to ApG dinucleotides to reduce the O/E CpG density.
B) Fold induction in Firefly Luciferase activity by artificial Banp promoters with reducing O/E CpG density over scrambled motif controls following transient transfection into mESCs. The activity of the promoter highlighted with the grey star can be compared to the activity of the same promoter in FIG. 2 after stable genomic integration. Shown is the average of three biological replicates of at least one clone with the standard deviation. The numbers 0 to 100 indicate the percentage of CpGs mutated to ApGs.

FIG. 2: Luciferase reporter activity is suppressed upon stable genomic integration of an artificial Banp promoter with 50% reduced CpG density
A) Artificial Banp promoters with three intact or scrambled motifs containing different CpG densities were stably integrated into the β-globin locus in mESCs.
B) Firefly-luciferase reporter activity of the stably integrated promoters. The activity of the promoter highlighted with the grey star can be compared to the activity of the same promoter in FIG. 1 after transient transfection. The numbers 0 to 100 indicate the percentage of CpGs mutated to ApGs. Plotted is the average of four biological replicates. The error bar represents the standard deviation.

### DETAILED DESCRIPTION OF THE INVENTION

The present inventors have previously found that a thus far orphaned regulatory motif in mammalian, when bound by protein BANP, acts as a strong transcriptional activator, also of CpG island promoters. This strong activator effect is synergistically increased when more than one copy of the motif is present in front of a heterologous transgene.

Upon further investigations, the present inventors found that the number of CpG sites in the vicinity of the orphaned regulatory motif influences the activity of the motif. This effect can be used to regulate the expression of genes operatively linked to said motif. For example, an expression vector can harbor more than one heterologous transgene, each under the control of its respective BANP motif, but with a different CpG density around each of these motives. This will result in a different, controlled, expression of each of the transgenes, despite being on the same vector and controlled by the same motif bound by the same transcription factor. Another advantage of the present invention is that CpG-rich motives are usually highly controlled by the cells and will lead to a switching off of the expression of the transgene if the construct is accidentally incorporated into the genome of the host cell.

The present invention hence provides an isolated nucleic acid molecule comprising more than 220 bp, one or more copy of a sequence selected from the group of SEQ ID NO:1, SEQ ID NO:2 and SEQ ID NO:3, and a CpG Observed over Estimated ratio (O/E ratio) larger than 0.6 in the N base pairs (bp) preceding and/or in the N bp following said one or more copy of a sequence selected from the group of SEQ ID NO:1, SEQ ID NO:2 and SEQ ID NO:3, wherein the CpG O/E ratio is determined by counting the number of CpG dinucleotides in the N bp-long sequences surrounding the at least one or more copy of a sequence selected from the group of SEQ ID NO:1, SEQ ID NO:2 and SEQ ID NO:3 and calculating the O/E ratio by multiplying the counted number of CpG dinucleotides by N and dividing the result by the product of the number of C and number of G present in the N bp (N*CpG/(C*G)), wherein N is between 50 and 1000 and is the length, in bp, of the sequence immediately preceding or immediately following said one or more copy of a sequence selected from the group of SEQ ID NO:1, SEQ ID NO:2 and SEQ ID NO:3. For the sake of clarity, it is stated that it is well known to the skilled person that C stands for a cytosine nucleotide, G stands for guanosine nucleotide and CpG (or CG) stands for 5′-C-phosphate-G-3′, i.e. cytosine and guanine separated by only one phosphate group (phosphate links any two nucleosides together in DNA).

In some embodiments, the CpGs are present in the 50-1000 bp immediately preceding the one or more copy of a sequence selected from the group of SEQ ID NO:1, SEQ ID NO:2 and SEQ ID NO:3, and an heterologous transgene is situated, immediately or not, after said one or more copy of the sequence selected from the group of SEQ ID NO:1, SEQ ID NO:2 and SEQ ID NO:3.

In some embodiments, the CpGs are present in the 50-1000 bp immediately following the one or more copy of a sequence selected from the group of SEQ ID NO:1, SEQ ID NO:2 and SEQ ID NO:3.

In some embodiments, In some embodiments, the CpGs are present in the 50-1000 bp immediately preceding and on the 50-1000 bp following the one or more copy of a sequence selected from the group of SEQ ID NO:1, SEQ ID NO:2 and SEQ ID NO:3.

In some embodiments, N is approximatively 50. In some embodiments, N is approximatively 100. In some embodiments, N is approximatively 150. In some embodiments, N is approximatively 200. In some embodiments, N is approximatively 250. In some embodiments, N is approximatively 500. In some embodiments, N is approximatively 800. In some embodiments, N is approximatively 1000.

The nucleic acid sequence of the sequence of the invention are:

SEQ ID NO: 1
BMYCGCGRBV
SEQ ID NO: 2
YMYCGCGRKV
SEQ ID NO: 3
TCTCGCGAGA

In some embodiments, the isolated nucleic acid of the invention further comprises, operably linked to a constitutive promoter or to an inducible promoter, a further sequence encoding for protein BANP, or for an active fragment or variant thereof.

In some embodiments, the heterologous transgene of the isolated nucleic acid of the invention is a chimeric antigen receptor.

The present invention also provides a vector comprising the isolated nucleic acid of the invention. In some embodiments, this vector is a plasmid, DNA vector, RNA vector, viral vector, adenoviral vector, adenoassociated viral vector, lentiviral vector, retroviral vector, gamma retroviral vector, or HSV vector. In some embodiments, the isolated nucleic acid of the invention is less than 8 Kb. In some embodiments, the isolated nucleic acid of the invention is less than 5 Kb.

The present invention also provides a kit or composition comprising an isolated nucleic acid of the invention and a second isolated nucleic molecule comprising a sequence encoding for protein BANP, or for an active fragment or variant thereof, operably linked to a constitutive promoter or to an inducible promoter. In such a kit, the isolated nucleic acids of the invention can be either within the same vector or within different vectors.

The present invention also provides the use of an isolated nucleic acid of the invention, a vector of the invention or a kit of the invention or composition of the invention for the, preferably transient, expression in vitro, ex vivo or in vivo of the heterologous transgene in a cell. In some embodiments, this use increases the expression of the heterologous transgene by a factor greater than two as compared to the expression of the heterologous transgene when operatively linked to a single copy of SEQ ID NO:1, SEQ ID NO:2 or SEQ ID NO:3 under the same conditions. In some embodiments, the expression of the heterologous transgene is measured by reporter gene activity, reporter gene fluorescence, quantitative reverse transcriptase PCR or genomics approaches such as RNA sequencing.

The present invention further provides a method of producing, in vitro, ex vivo or in vivo, a heterologous transgene in a cell by introducing any of the isolated nucleic acids of the invention or the vectors of the invention of claims the cell, culturing this cell (or cell population), and purifying the recombinantly expressed heterologous transgene. In some embodiments, the cell is a stem cell.

The present invention also provides the isolated cell comprising the isolated nucleic acid of the invention. In this cell, or cells, the isolated nucleic acid sequence comprising at least two copies of a sequence selected from the group of SEQ ID NO:1, SEQ ID NO:2 and/or SEQ ID NO:3 and the heterologous transgene can be stably integrated into the genome of said cell.

As used herein, the term “promoter” refers to any cis-regulatory elements, including enhancers, silencers, insulators and promoters. A promoter is a region of DNA that is generally located upstream (towards the 5′ region) of the gene that is needed to be transcribed. The promoter permits the proper activation or repression of the gene which it controls. In the context of the present invention, the promoters lead to the specific expression of genes operably linked to them in the cells expressing glial fibrillary acidic protein. “Specific expression” of an exogenous gene, also referred to as “expression only in a certain type of cell” means that at least more than 75%, preferably more than 85%, more that 90% or more than 95%, of the cells expressing the exogenous gene of interest are of the type specified, i.e. cells expressing glial fibrillary acidic protein in the present case.

Expression cassettes are typically introduced into a vector that facilitates entry of the expression cassette into a host cell and maintenance of the expression cassette in the host cell. Such vectors are commonly used and are well known to those of skill in the art. Numerous such vectors are commercially available, e.g., from Invitrogen, Stratagene, Clontech, etc., and are described in numerous guides, such as Ausubel, Guthrie, Strathem, or Berger, all supra. Such vectors typically include promoters, polyadenylation signals, etc. in conjunction with multiple cloning sites, as well as additional elements such as origins of replication, selectable marker genes (e.g., LEU2, URA3, TRP 1, HIS3, GFP), centromeric sequences, etc. For the sake of clarity, it is immediately evident for the skilled person that the present invention also includes the isolated nucleic acid having the sequences that are complementary to those defined in the claims.

Suitable viral vectors for the invention are well-known in the art. For instance an AAV, a PRV or a lentivirus, are suitable to target and deliver genes to cells.

As used herein, the term “animal” is used herein to include all animals. In some embodiments of the invention, the non-human animal is a vertebrate. Examples of animals are human, mice, rats, cows, pigs, horses, chickens, ducks, geese, cats, dogs, etc. The term “animal” also includes an individual animal in all stages of development, including embryonic and fetal stages. A “genetically-modified animal” is any animal containing one or more cells bearing genetic information altered or received, directly or indirectly, by deliberate genetic manipulation at a sub-cellular level, such as by targeted recombination, microinjection or infection with recombinant virus. The term “genetically-modified animal” is not intended to encompass classical crossbreeding or in vitro fertilization, but rather is meant to encompass animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA. The term “germ-line genetically-modified animal” refers to a genetically-modified animal in which the genetic alteration or genetic information was introduced into germline cells, thereby conferring the ability to transfer the genetic information to its offspring. If such offspring in fact possess some or all of that alteration or genetic information, they are genetically-modified animals as well.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene, or not expressed at all.

The genes used for altering a target gene may be obtained by a wide variety of techniques that include, but are not limited to, isolation from genomic sources, preparation of cDNAs from isolated mRNA templates, direct synthesis, or a combination thereof.

A type of target cells for transgene introduction is the ES cells. ES cells may be obtained from pre-implantation embryos cultured in vitro and fused with embryos (Evans et al. (1981), Nature 292:154-156; Bradley et al. (1984), Nature 309:255-258; Gossler et al. (1986), Proc. Natl. Acad. Sci. USA 83:9065-9069; Robertson et al. (1986), Nature 322:445-448; Wood et al. (1993), Proc. Natl. Acad. Sci. USA 90:4582-4584). Transgenes can be efficiently introduced into the ES cells by standard techniques such as DNA transfection using electroporation or by retrovirus-mediated transduction. The resultant transformed ES cells can thereafter be combined with morulas by aggregation or injected into blastocysts from a non-human animal. The introduced ES cells thereafter colonize the embryo and contribute to the germline of the resulting chimeric animal (Jaenisch (1988), Science 240:1468-1474). The use of gene-targeted ES cells in the generation of gene-targeted genetically-modified mice was described 1987 (Thomas et al. (1987), Cell 51:503-512) and is reviewed elsewhere (Frohman et al. (1989), Cell 56:145-147; Capecchi (1989), Trends in Genet. 5:70-76; Baribault et al. (1989), Mol. Biol. Med. 6:481-492; Wagner (1990), EMBO J. 9:3025-3032; Bradley et al. (1992), Bio/Technology 10:534-539).

Techniques are available to inactivate or alter any genetic region to any mutation desired by using targeted homologous recombination to insert specific changes into chromosomal alleles.

As used herein, a “targeted gene” is a DNA sequence introduced into the germline of a non-human animal by way of human intervention, including but not limited to, the methods described herein. The targeted genes of the invention include DNA sequences which are designed to specifically alter cognate endogenous alleles.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a pro-protein which can be activated by cleavage of the pro-protein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, denivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. I-12 (1983); Seifter et al; Meth Enzymol 182:626-646 (1990); Rattan et al., Ann NY Acad Sci 663:48-62 (1992).) A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.) Species homologs may be isolated and identified by making suitable probes or primers from the sequences provided herein and screening a suitable nucleic acid source for the desired homologue.

“Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty−1, Joining Penalty−30, Randomization Group Length=0, Cutoff Score=I, Gap Penalty−5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty−I, Joining Penalty=20, Randomization Group Length=0, Cutoff Score=I, Window Size=sequence length, Gap Penalty−5, Gap Size Penalty−0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

As used herein, an isolated nucleic acid comprising a “heterologous nucleic acid sequence” or a “heterologous transgene” refers to an isolated nucleic acid comprising a portion (i.e., the heterologous nucleic acid portion) that is not normally found operably linked to the rest of the isolated nucleic acid in a natural context. For instance, the heterologous nucleic acid may comprise a nucleic acid sequence not originally found in a cell, bacterial cell, virus, or organism from which other components of the isolated nucleic acid (e.g., the promoter) naturally derive or where the other components of the isolated nucleic acid (e.g., the promoter) are not naturally found operatively linked with the heterologous nucleic acid in the cell, bacterial cell, virus, or organism. In some embodiments, the heterologous nucleic acid sequence encodes a human protein. In some embodiments, the heterologous nucleic acid sequence encodes an RNA sequence, e.g., a shRNA.

A DNA sequence or DNA polynucleotide sequence that “encodes” a particular RNA is a sequence of DNA that is capable of being transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A DNA sequence or DNA polynucleotide sequence may also “encode” a particular polypeptide or protein sequence, wherein, for example, the DNA directly encodes an mRNA that can be translated into the polypeptide or protein sequence. A “protein coding sequence” or a sequence that encodes a particular protein or polypeptide is a nucleic acid sequence that is capable of being transcribed into mRNA (in the case of DNA) and translated (in the case of mRNA) into a polypeptide in vitro or in vivo when placed under the control of appropriate regulatory sequences. The boundaries of the coding sequence may be determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., a short hairpin RNA) or a coding sequence (e.g., PGRN) and/or regulate translation of an encoded polypeptide.

The terms “polyadenylation (polyA) signal sequence” and “polyadenylation sequence” refer to a regulatory element that provides a signal for transcription termination and addition of an adenosine homopolymeric chain to the 3′ end of an RNA transcript. The polyadenylation signal may comprise a termination signal (e.g., an AAUAAA sequence or other non-canonical sequences) and optionally flanking auxiliary elements (e.g., a GU-rich element) and/or other elements associated with efficient cleavage and polyadenylation. The polyadenylation sequence may comprise a series of adenosines attached by polyadenylation to the 3′ end of an mRNA. Specific polyA signal sequences may include the poly(A) signal of Table 1 (SEQ ID NO:5). In some embodiments, DNA regulatory sequences or control elements are tissue-specific regulatory sequences.

The term “post-transcriptional regulatory element” (“PRE”) refers to one or more regulatory elements that, when transcribed into mRNA, regulate gene expression at the level of the mRNA transcript. Examples of such post-transcriptional regulatory elements may include sequences that encode micro-RNA binding sites, RNA binding protein binding sites, etc.. Examples of post-transcriptional regulatory element that may be used with the viral vectors disclosed herein include the woodchuck hepatitis post-transcriptional regulatory element (WPRE), the hepatitis post-transcriptional regulatory element (HPRE).

The term “intron” refers to nucleic acid sequence(s), e.g., those within an open reading frame, that are noncoding for one or more amino acids of a protein expressed from the nucleic acid. Intronic sequences may be transcribed from DNA into RNA, but may be removed before the protein is expressed, e.g., through splicing. In some embodiments, intron sequences are added to a heterologous nucleic acid sequence to increase overall efficiency and output of gene expression. Examples of introns that may be used with the viral vectors disclosed herein include the SV40 intron, the betaglobin intron, the chicken beta-actin intron etc..

As used herein, processes conducted “in vitro” refer to processes which are performed outside of the normal biological environment, for example, studies performed in a test tube, a flask, a petri dish, in artificial culture medium. Processes conducted “in vivo” refer to processes performed within living organisms or cells. for example, studies performed in cell cultures or in mice. Processes performed “ex vivo” refer to proceses done in or on tissue from an organism in an external environment, e.g., with minimal alteration of natural conditions, e.g., allowing for manipulation of an organism's cells or tissues under more controlled conditions than may be possible in in vivo experiments.

The term “naturally-occurring” or “unmodified” as used herein as applied to, e.g., a nucleic acid, a polypeptide, a cell, or an organism, is one found in nature. For example, a polypeptide or polynucleotide sequence that is present in an organism (such as a virus) is naturally occurring whether present in that organism or isolated from one or more components of the organism.

In some embodiments, a “vector” is any genetic element (e.g., DNA, RNA, or a mixture thereof) that contains a nucleic acid of interest that is capable of being expressed in a host cell, e.g., a nucleic acid of interest within a larger nucleic acid sequence or structure suitable for delivery to a cell, tissue, and/or organism, such as a plasmid, phage, transposon, cosmid, chromosome, virus, virion, etc. For instance, a vector may comprise an insert (e.g., a heterologous nucleic acid encoding a gene to be expressed or an open reading frame of that gene) and one or more additional elements, e.g., elements suitable for delivering or controlling expression of the insert. The vector may be capable of replication and/or expression, e.g., when associated with the proper control elements, and it may be capable of transferring genetic information between cells. In some embodiments, a vector may be a vector suitable for expression in a host cell, e.g, an AAV vector. In some embodiments, a vector may be a plasmid suitable for expression and/or replication, e.g., in a cell or bioreactor. In some embodiments, vectors designed specifically for the expression of a heterologous nucleic acid sequence, e.g., a heterologous nucleic acid encoding a protein of interest, shRNA, and the like, in the target cell may be referred to as expression vectors, and generally have a promoter sequence that drives expression of the heterologous nucleic acid sequence. In other embodiments, vectors, e.g., transcription vectors, may be capable of being transcribed but not translated: they can be replicated in a target cell but not expressed. Transcription vectors may be used to amplify their insert.

The term “expression vector” refers to a vector comprising a polynucleotide comprising expression control sequences operatively linked to a nucleotide sequence to be expressed. An expression vector may comprise sufficient cis-acting elements for expression, alone or in combination with other elements for expression supplied by the host cell or in an in vitro expression system. Expression vectors include, e.g., cosmids, plasmids (e.g., naked or contained in liposomes) and viruses (e.g., lentiviruses, retroviruses, adenoviruses, and adeno-associated viruses) that incorporate the recombinant polynucleotide.

The term “plasmid” refers to a nonchromosomal (and typically double-stranded) DNA sequence comprising an intact “replicon” such that the plasmid is replicated in a host cell. A plasmid may be a circular nucleic acid. When the plasmid is placed within a unicellular organism, the characteristics of that organism are changed or transformed as a result of the DNA of the plasmid. For example, a plasmid carrying the gene for tetracycline resistance (TcR) transforms a cell previously sensitive to tetracycline into one which is resistant to it. The term “recombinant virus” as used herein is intended to refer to a non-wild-type and/or artificially produced recombinant virus (e.g., a parvovirus, adenovirus, lentivirus or adeno-associated virus etc.) that comprises a gene or other heterologous nucleic acid. The recombinant virus may comprise a recombinant viral genome (e.g. comprising a nucleic acid encoding the gene of interest) packaged within a viral (e.g.: AAV) capsid. A specific type of recombinant virus may be a “recombinant adeno-associated virus”, or “rAAV”. The recombinant viral genome packaged in the viral capsid may be a viral vector. In some embodiments, the recombinant viruses disclosed herein comprise viral vectors. Examples of viral vectors include but are not limited to an adeno-associated viral (AAV) vector, a chimeric AAV vector, an adenoviral vector, a retroviral vector, a lentiviral vector, a DNA viral vector, a herpes simplex viral vector, a baculoviral vector, or any mutant or derivative thereof.

In another embodiment, the term “transfection” is used to refer to the uptake of foreign DNA by a cell, such that the cell has been “transfected” once the exogenous DNA has been introduced inside the cell membrane. See, e.g., Graham et al., (1973) Virology, 52:456; Sambrook et al., (1989) Molecular Cloning, a laboratory manual, Cold Spring Harbor Laboratories, New York; Davis et al., (1986) Basic Methods in Molecular Biology, Elsevier; Chu et al., (1981) Gene, 13:197. Such techniques can be used to introduce one or more exogenous DNA moieties into suitable host cells. In some embodiments, the term “transduction” is used to refer to the uptake of foreign DNA by a cell, where the foreign DNA is provided by a virus or a viral vector. Consequently, a cell has been “transduced” when exogenous DNA has been introduced inside the cell membrane. In some embodiments, the term “transformation” is used to refer to the uptake of foreign DNA by bacterial cells.

As used herein, the term “cell line” refers to a population of cells capable of continuous or prolonged growth and division in vitro. In certain circumstances, spontaneous or induced changes can occur in karyotype during storage or transfer of such clonal populations. Therefore, cells derived from the cell line referred to may not be precisely identical to the ancestral cells or cultures, and the cell line referred to includes such variants.

The term “operably linked” refers to a functional relationship between two or more polynucleotide (e.g., DNA) segments. Typically, the term refers to the functional relationship of a transcriptional regulatory sequence and a sequence to be transcribed. For example, a promoter or enhancer sequence is operably linked to a coding sequence if it, e.g., stimulates or modulates the transcription of the coding sequence in an appropriate host cell or other expression system. Generally, promoter transcriptional regulatory sequences that are operably linked to a sequence are contiguous to that sequence or are separated by short spacer sequences, i.e., they are cis-acting. However, some transcriptional regulatory sequences, such as enhancers, need not be physically contiguous or located in close proximity to the coding sequences whose transcription they enhance.

As used herein, the term “AAV vector” refers to a vector derived from or comprising one or more nucleic acid sequences derived from an adeno-associated virus serotype, including without limitation, an AAV-1, AAV-2, AAV-3, AAV-4, AAV-5, AAV-6, AAV-7, AAV-8 or AAV-9 viral vector. AAV vectors may have one or more of the AAV wild-type genes deleted in whole or part, e.g., the rep and/or cap genes, while retaining, e.g., functional flanking inverted terminal repeat (“ITR”) sequences. In some embodiments, an AAV vector may be packaged in a protein shell or capsid, e.g., comprising one or more AAV capsid proteins, which may provide a vehicle for delivery of vector nucleic acid to the nucleus of target cells. In some embodiments, an AAV vector comprises one or more AAV ITR sequences (e.g., AAV2 ITR sequences). In some embodiments, an AAV vector comprises one or more AAV ITR sequences (e.g., AAV2 ITR sequences) but does not contain any additional viral nucleic acid sequence. Embodiments of these vector constructs are provided, e.g., in WO/2019/094253 (PCT/US2018/058744), which is incorporated herein by reference in its entirety.

In some embodiments, an “scAAV” is a self-complementary adeno-associated virus (scAAV). scAAV is termed “self-complementary” because at least a portion of the vector (e.g., at least a portion of the coding region) of the scAAV forms an intra-molecular double-stranded DNA. In some embodiments, the rAAV is a scAAV. In some embodiments, a viral vector is engineered from a naturally occurring adeno-associated virus (AAV) to provide an scAAV for use in gene therapy. Embodiments of these vector constructs and methods of preparing and purifying them are provided, e.g., in WO/2019/094253 (PCT/US2018/058744), which is incorporated herein by reference in its entirety.

As used herein, an “virus” or “irion” indicates a viral particle, comprising a viral vector, e.g., alone or in combination with one or more additional components such as one or more viral capsids. For instance, an AAV virus may comprise, e.g., a linear, single-stranded AAV nucleic acid genome associated with an AAV capsid protein coat.

In some embodiments, terms such as “virus,” “virion,” “AAV virus,” “recombinant AAV virion,” “rAAV virion,” “AAV vector particle,” “full capsids,” “full particles,” and the like refer to infectious, replication-defective virus, e.g., those comprising an AAV protein shell encapsidating a heterologous nucleotide sequence of interest, e.g., in a viral vector which is flanked on one or both sides by AAV ITRs. A rAAV virion may be produced in a suitable host cell which comprises sequences, e.g., one or more plasmids, specifying an AAV vector, alone or in combination with nucleic acids encoding AAV helper functions and accessory functions (such as cap genes), e.g., on the same or additional plasmids. In some embodiments, the host cell is rendered capable of encoding AAV polypeptides that provide for packaging the AAV vector (containing a recombinant nucleotide sequence of interest) into infectious recombinant virion particles for subsequent gene delivery.

The terms “inverted terminal repeat” or “ITR” refer to a stretch of nucleotide sequences that can form a T-shaped palindromic structure, e.g., in adeno-associated viruses (AAV) and/or recombinant adeno-associated viral vectors (rAAV). Muzyczka et al., (2001) Fields Virology, Chapter 29, Lippincott Williams & Wilkins. In recombinant AAV vectors, these sequences may play a functional role in genome packaging and in second-strand synthesis.

The term “host cell” denotes a cell comprising an exogenous nucleic acid of interest, for example, one or more microorganism, yeast cell, insect cell, or mammalian cell. For instance, the host cell may comprise an AAV helper construct, an AAV vector plasmid, an accessory function vector, and/or other transfer DNA. The term includes the progeny of the original cell which has been transfected. The progeny of a single parental cell may not necessarily be completely identical in morphology or in genomic or total DNA complement as the original parent, due to natural, accidental, or deliberate mutation.

The term “AAV helper function” refers to an AAV-derived coding sequences which can be expressed to provide AAV gene products, e.g., those that function in trans for productive AAV replication. For instance, AAV helper functions may include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions may be used herein to complement AAV functions in trans that are missing from AAV vectors.

The term “AAV helper construct” refers generally to a nucleic acid molecule that includes nucleotide sequences providing or encoding proteins or nucleic acids that provide AAV functions deleted from an AAV vector, e.g. a vector for delivery of a nucleotide sequence of interest to a target cell or tissue. AAV helper constructs are commonly used to provide transient expression of AAV rep and/or cap genes to complement missing AAV functions for AAV replication. Typically, helper constructs lack AAV ITRs and can neither replicate nor package themselves. AAV helper constructs may be in the form of a plasmid, phage, transposon, cosmid, virus, or virion. A number of AAV helper constructs have been disclosed, such as the commonly used plasmids pAAV/Ad and plM29+45 which encode both Rep and Cap expression products. See, e.g., Samulski et al., (1989) J. Virol., 63:3822-3828; McCarty et al., (1991) J. Virol., 65:2936-2945. A number of other vectors have been disclosed which encode Rep and/or Cap expression products. See, e.g., U.S. Pat. Nos. 5,139,941 and 6,376,237. Embodiments of these vector constructs and methods of preparing and purifying them are provided, e.g., in WO/2019/094253 (PCT/US2018/058744), which is incorporated herein by reference in its entirety.

“Label” refers to agents that are capable of providing a detectable signal, either directly or through interaction with one or more additional members of a signal producing system. Labels that are directly detectable and may find use in the invention include fluorescent labels. Specific fluorophores include fluorescein, rhodamine, BODIPY, cyanine dyes and the like.

A “fluorescent label” refers to any label with the ability to emit light of a certain wavelength when activated by light of another wavelength.

“Fluorescence” refers to any detectable characteristic of a fluorescent signal, including intensity, spectrum, wavelength, intracellular distribution, etc.

“Detecting” fluorescence refers to assessing the fluorescence of a cell using qualitative or quantitative methods. In some of the embodiments of the present invention, fluorescence will be detected in a qualitative manner. In other words, either the fluorescent marker is present, indicating that the recombinant fusion protein is expressed, or not. For other instances, the fluorescence can be determined using quantitative means, e.g., measuring the fluorescence intensity, spectrum, or intracellular distribution, allowing the statistical comparison of values obtained under different conditions. The level can also be determined using qualitative methods, such as the visual analysis and comparison by a human of multiple samples, e.g., samples detected using a fluorescent microscope or other optical detector (e.g., image analysis system, etc.). An “alteration” or “modulation” in fluorescence refers to any detectable difference in the intensity, intracellular distribution, spectrum, wavelength, or other aspect of fluorescence under a particular condition as compared to another condition. For example, an “alteration” or “modulation” is detected quantitatively, and the difference is a statistically significant difference. Any “alterations” or “modulations” in fluorescence can be detected using standard instrumentation, such as a fluorescent microscope, CCD, or any other fluorescent detector, and can be detected using an automated system, such as the integrated systems, or can reflect a subjective detection of an alteration by a human observer.

The “green fluorescent protein” (GFP) is a protein, composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostablility and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted ε), also known as its optical cross section of 9.13×10−21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm. As used herein, the singular forms “a”, “an,” and “the” include plural reference unless the context clearly dictates otherwise.

A “virus” is a sub-microscopic infectious agent that is unable to grow or reproduce outside a host cell. Each viral particle, or virion, consists of genetic material, DNA or RNA, within a protective protein coat called a capsid. The capsid shape varies from simple helical and icosahedral (polyhedral or near-spherical) forms, to more complex structures with tails or an envelope. Viruses infect cellular life forms and are grouped into animal, plant and bacterial types, according to the type of host infected.

The term “transsynaptic virus” as used herein refers to viruses able to migrate from one neurone to another connecting neurone through a synapse. Examples of such transsynaptic virus are rhabodiviruses, e.g. rabies virus, and alphaherpesviruses, e.g. pseudorabies or herpes simplex virus. The term “transsynaptic virus” as used herein also encompasses viral sub-units having by themselves the capacity to migrate from one neurone to another connecting neurone through a synapse and biological vectors, such as modified viruses, incorporating such a sub-unit and demonstrating a capability of migrating from one neurone to another connecting neurone through a synapse.

Transsynaptic migration can be either anterograde or retrograde. During a retrograde migration, a virus will travel from a postsynaptic neuron to a presynaptic one. Accordingly, during anterograde migration, a virus will travel from a presynaptic neuron to a postsynaptic one.

Homologs refer to proteins that share a common ancestor. Analogs do not share a common ancestor, but have some functional (rather than structural) similarity that causes them to be included in a class (e.g. trypsin like serine proteinases and subtilisin's are clearly not related—their structures outside the active site are completely different, but they have virtually geometrically identical active sites and thus are considered an example of convergent evolution to analogs).

There are two subclasses of homologs—orthologs and paralogs. Orthologs are the same gene (e.g. cytochome ‘c’), in different species. Two genes in the same organism cannot be orthologs. Paralogs are the results of gene duplication (e.g. hemoglobin beta and delta). If two genes/proteins are homologous and in the same organism, they are paralogs.

As used herein, the term “disorder” refers to an ailment, disease, illness, clinical condition, or pathological condition.

As used herein, the term “pharmaceutically acceptable carrier” refers to a carrier medium that does not interfere with the effectiveness of the biological activity of the active ingredient, is chemically inert, and is not toxic to the patient to whom it is administered.

As used herein, the term “pharmaceutically acceptable derivative” refers to any homolog, analog, or fragment of an agent, e.g. identified using a method of screening of the invention, that is relatively non-toxic to the subject.

The term “therapeutic agent” refers to any molecule, compound, or treatment, that assists in the prevention or treatment of disorders, or complications of disorders.

Compositions comprising such an agent formulated in a compatible pharmaceutical carrier may be prepared, packaged, and labeled for treatment.

If the complex is water-soluble, then it may be formulated in an appropriate buffer, for example, phosphate buffered saline or other physiologically compatible solutions.

Alternatively, if the resulting complex has poor solubility in aqueous solvents, then it may be formulated with a non-ionic surfactant such as Tween, or polyethylene glycol. Thus, the compositions and their physiologically acceptable solvates may be formulated for administration by inhalation or insufflation (either through the mouth or the nose) or oral, buccal, parenteral, rectal administration or, in the case of tumors, directly injected into a solid tumor.

The compositions may be formulated for parenteral administration by injection, e.g., by bolus injection or continuous infusion. Formulations for injection may be presented in unit dosage form, e.g., in ampoules or in multi-dose containers, with an added preservative.

The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions may also be formulated as a topical application, such as a cream or lotion. In addition to the formulations described previously, the compositions may also be formulated as a depot preparation. Such long acting formulations may be administered by implantation (for example, intraocular, subcutaneous or intramuscular) or by intraocular injection.

Thus, for example, the composition may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The invention also provides kits for carrying out the therapeutic regimens of the invention. Such kits comprise in one or more containers therapeutically or prophylactically effective amounts of the compositions in pharmaceutically acceptable form.

The composition in a vial of a kit may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit further comprises a needle or syringe, preferably packaged in sterile form, for injecting the complex, and/or a packaged alcohol pad. Instructions are optionally included for administration of compositions by a clinician or by the patient.

Protein BANP, also known as BTG3 Associated Nuclear Protein, Scaffold/Matrix-Associated Region-1-Binding Protein, BEN Domain-Containing Protein 1, Protein BANP, BEND1, SMAR1, Btg3-Associated Nuclear Protein, BEN Domain Containing 1, or SMARBP1, is a protein that in humans is encoded by the BANP gene (HGNC: 13450 Entrez Gene: 54971 Ensembl: ENSG00000172530 OMIM: 611564 UniProtKB: Q8N9N5). It is a member of the human gene family, “BEN-domain containing”, which includes eight other genes: BEND2, BEND3, BEND4, BEND5, BEND6, BEND7, NACC1 (BEND8), and NACC2 (BEND9).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Examples

Dual Luciferase Reporter Assay after Transient Transfection

Three Banp motif and scrambled controls imbedded in an artificial promoter sequence with various CpG dinucleotide density (0%, 25%, 50%, 75%, or 100% mutated CpGs) were cloned upstream of a Firefly Luciferase gene. The Firefly Luciferase plasmid was co-transfected with a Renilla Luciferase control reporter plasmid (10:1) into mouse embryonic stem cells (mESCs) in a 24 well plate using Lipofectamine-2000 (Thermo Fisher Scientific, L3000008). After 24 hours the Luciferase Assay System (Promega E1500) was performed. Cells were washed once with PBS and lysed with Passive Lysis Buffer (PLB, 100 ul) with gentle agitation for 15 min at room temperature. Luciferase Assay Reagent II (LAR II, 100 ul) was dispensed into the appropriate number of wells in a 96 well luminometer plate. Luminometer programmed to perform a 2 sec premeasurement delay, followed by a 10 sec measurement period for each reporter assay. Carefully transfered 20 μl of cell Iysate into the luminometer plate containing LAR II, mixed by pipetting 3 times up and down, then measured the Firefly luciferase activity. The sample plate was removed from the luminometer, Stop & Glo Reagent (100 μl) added and vortexed briefly to mix. Replaced the samples in the luminometer, and measured the Renilla luciferase activity. Firefly luciferase activity was normalized to Renilla luciferase activity and then the fold increase in Firefly luciferase activity for Banp motif containing constructs was determined relative to scrambled control motif containing construct.

Stable Genomic Integration of Banp Promoters and Luciferase Reporter Assay

The artificial Banp promoter-luciferase constructs with three intact or scrambled Banp motifs were stably integrated into the β-globin locus of mESCs. Four separate clones baring each of the Banp promoters were selected and 250,000 cells were plated 24 hours before performing the Luciferase Assay System (Promega E1500). In short, cells were lysed in 250 μl 1×PLB, incubated with shaking for 10 min, and transferred into tubes on ice. The cells were vortexed for 1 sec, spun down for 15 sec at room temperature, and the supernatant was transferred to new tubes on ice. Cell lysates (20 μl) were aliquoted in duplicate into to a 96-well plate, 100 μl Luciferase Assay Reagent was added per well, and the mix pipetted up and down three times. The firefly-luciferase signal was measured with a luminometer for 1 s per well without delay.

1. An isolated nucleic acid molecule comprising
a. more than 220 bp,
b. one or more copy of a sequence selected from the group of SEQ ID NO:1, SEQ ID NO:2 and SEQ ID NO:3, and
c. a CpG Observed over Estimated ratio (O/E ratio) larger than 0.6 in the N base pairs (bp) preceding and/or in the N bp following said one or more copy of a sequence selected from the group of SEQ ID NO:1, SEQ ID NO:2 and SEQ ID NO:3,
wherein the CpG O/E ratio is determined by counting the number of CpG dinucleotides in the N bp-long sequences surrounding the at least one or more copy of a sequence selected from the group of SEQ ID NO:1, SEQ ID NO:2 and SEQ ID NO:3 and calculating the O/E ratio by multiplying the counted number of CpG dinucleotides by N and dividing the result by the product of the number of C and number of G present in the N bp (N*CpG/(C*G)), wherein N is between 50 and 1000 and is the length, in bp, of the sequence immediately preceding or immediately following said one or more copy of a sequence selected from the group of SEQ ID NO:1, SEQ ID NO:2 and SEQ ID NO:3.

2. The isolated nucleic acid of claim 1 further comprising a heterologous transgene.

3. The isolated nucleic acid of claim 1 further comprising, operably linked to a constitutive promoter or to an inducible promoter, a further sequence encoding for protein BANP, or for an active fragment or variant thereof.

4. The isolated nucleic acid of claim 2, wherein the heterologous transgene is a chimeric antigen receptor.

5. A vector comprising the isolated nucleic acid of claim 1.

6. The vector of claim 5, wherein said vector is a plasmid, DNA vector, RNA vector, viral vector, adenoviral vector, adenoassociated viral vector, lentiviral vector, retroviral vector, gamma retroviral vector, or HSV vector.

7. A kit or composition comprising an isolated nucleic acid of claim 1 and a second isolated nucleic molecule comprising a sequence encoding for protein BANP, or for an active fragment or variant thereof, operably linked to a constitutive promoter or to an inducible promoter.

8. A kit or composition according to claim 7 wherein both isolated nucleic acids are within the same vector.

9. A kit or composition according to claim 7 comprising at least two vectors, wherein both isolated nucleic acids are within different vectors.

10. A method of expressing a heterologous transene comprising introducing the isolated nucleic acid of claim 1 into a cell.

11. The method of claim 10 wherein the expression of the heterologous transgene is increased by a factor greater than two as compared to the expression of the heterologous transgene when operatively linked to a single copy of SEQ ID NO:1, SEQ ID NO:2 or SEQ ID NO:3 under the same conditions.

12. The method of claim 10, wherein said expression is measured by reporter gene activity, reporter gene fluorescence, quantitative reverse transcriptase PCR or genomics approaches such as RNA sequencing.

13. The method of claim 10 further comprising, culturing said cell, and purifying the recombinantly expressed heterologous transgene.

14. An isolated cell comprising the isolated nucleic acid of claim 1.

15. The cell of claim 14 wherein the isolated nucleic acid sequence comprising at least two copies of a sequence selected from the group of SEQ ID NO:1, SEQ ID NO:2 and/or SEQ ID NO:3 and the heterologous transgene is stably integrated into the genome of said cell.


SEQUENCE LISTING (3 sequences)

SEQ ID NO: 1 - DNA, 10 bp, Homo sapiens
bmycgcgrbv

SEQ ID NO: 2 - DNA, 10 bp, Homo sapiens
ymycgcgrkv

SEQ ID NO: 3 - DNA, 10 bp, Homo sapiens
tctcgcgaga